(19) United States
(12) Patent Application Publication
(10) Pub. No.: US 2010/0047117 A1
(43) Pub. Date: Feb. 25, 2010

(54) PROCESS FOR AIR PURIFICATION AND AIR PURIFIER DEVICE FOR ITS IMPLEMENTATION

(76) Inventor: Marie Charlotte Bernard, Tours (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/594,619

(22) PCT Filed: Apr. 7, 2008
(86) PCT No.: PCT/IB2008/000832

§ 371 Date: Oct. 5, 2009

(30) Foreign Application Priority Data
Apr. 5, 2007 (FR) 07 02573

(51) Int. Cl. A61L 9/00

(52) U.S. Cl. 422/4; 422/121

(57) ABSTRACT

This invention relates to the purification of indoor air using a method that essentially comprises forcing the air to be processed to flow through an active medium exposed to a germicidal radiation in the presence of a photocatalytic material contained therein, with a destructive action on the microorganisms optionally present in the air and retained in the active medium such as reduced and/or weakened due to the impact on wall members provided therefore in the active medium. A first active medium includes a mass made in the form of a three-dimensional network of walls with photocatalytic surfaces and located in the immediate vicinity of a germicidal radiation source. A second active medium includes a liquid mass in which particles with a photocatalytic surface are dispersed and submitted to a germicidal radiation. The liquid mass is agitated by feeding air therein in order to generate a vortex.

This invention concerns a method for purification of the inside air of a building as well as an air purification device that is designed for the implementation of such method.

The invention will find possible uses in all of the sectors in which there is a health requirement for controlling the ambient air quality, whether it is a matter of private premises or public facilities. It applies particularly but not exclusively to the purification of the inside air within the hospital sector and to other domains within the jurisdiction of health or hygiene, as well as to the purification of ambient air in all living spaces, in particular those where individuals suffering from respiratory ailments live, as well as also to the purification of air in all enclosed or semi-closed premises, for health reasons as well as for matters of comfort. In all of the environments of this type, it has the advantage of allowing the effective destruction of pollutants that can be contained in the air, knowing that the most negative pollutants that it is desired to eliminate under these circumstances are primarily organic pollutants, in the form of bacteria or other microorganisms.

To ensure the control of the air quality inside buildings, in particular in closed rooms, the invention calls for subjecting the air to be treated to a treatment that is carried out in a continuous loop by operating on air that is drawn off from the ambient atmosphere and that is sent back into the same atmosphere after treatment. This operating mode helps ensure the prevention of respiratory diseases and other ailments that are linked to air-borne infectious agents. It represents in particular an advantageous alternative to the renewal of air by ventilation in the rooms of health institutions. It is also particularly useful in domestic applications to combat the presence of bio-contaminants, for example the microorganisms such as viruses, bacteria, spores, lesser fungi or allergens such as scales of domestic animals, pollens, mite dirt, bacterial fragments, especially since the presence of these bio-contaminants is promoted by a confinement that is increasingly desired by measuring energy savings.

Among the ways to combat indoor pollution, in addition to the improvement of ventilation and/or limitation of the emission of pollutants, air purifier devices that use the photo-catalytic action. and whose objective is to truly destroy the organic compounds that are conveyed by air rather than to keep them trapped on a filter, are known. The catalyst of the destructive electronic process is in general titanium dioxide which, exposed to ultraviolet radiation, emits electrons that act to eliminate the organic compounds by oxidation.

Some of the preferred implementations of this invention are similar to this known technique, to the extent that use is also made of a photo-catalytic action under UV irradiation. The invention, however, calls for operating under conditions that lead to very significantly improved results, in particular due to a better control of the contact between the air to be treated and the catalytic surface, also due to a mechanical-type action that attacks the particles within the treatment environment itself with an action that destroys the organic material.

More specifically, the invention uses the combination of a photo-catalytic treatment that destroys organic pollutants with a process of trapping particles that is enhanced due to their having been reduced in size and/of weakened by having impacted against the solid wall elements that are present within the active medium used for the photo-catalytic treatment itself.

Subsequent to this disclosure, two major pathways for the implementation of the invention will be distinguished, depending on whether the treatment takes place in the dry phase or in the wet phase. It will be seen that in all cases, the invention preferably provides that the wall elements that are present in the active medium to accommodate impacts of particles suspended in air are also the substrate of the catalyst with an activating action for the radiation to which this environment is exposed. The beneficial result in the effectiveness of the treatment for the destruction of organic components present in the air is double: on the one hand, this allows a greater density of the active matter in the treatment medium, since the active material is the same for the photo-catalysis and for the impact; on the other hand, the impact of the organic molecules on the photo-catalyst walls leads to trapping them very close to the location of the germicidal photo-catalytic action even when they have just undergone the mechanical destructive action of the impact.

As much for the impact as for the photo-catalytic action or else for the trapping of pollutant components, another aspect is that the solid elements of the active medium are in divided form, hence the form advantageously of dispersed particles, and that they are being continuously renewed on the path of the air to be treated, which accounts for the importance of the mixing, which mixing can be imposed either on the air circulating through the mass of the active medium, or on the treatment environment as a whole, or as well in the active medium itself when made of particles dispersed in the treatment environment.

According to another characteristic of the invention that is common to the dry or wet variant embodiments, the conditions of the photo-catalytic treatment are advantageously determined to exploit a germicidal action that is suitable for the radiation that is used, in addition to the photo-catalytic action. The selection of titanium dioxide as catalyst is then particularly suitable in that it is used advantageously under irradiation by an ultraviolet radiation in the UVC range. In the range of 10 to 280 nanometers of wavelength, the preferred wavelength for the direct germicidal action is located at about 260 nanometers.

Hereinafter, the first thing to be considered is an embodiment of an air purification device according to the invention that is designed for dry-phase operation. The photo-catalyst is then distributed in a porous mass arranged along a radiation source that is advantageously longitudinal to ensure that air circulates in the longitudinal direction of the porous mass.

In a preferred process according to the invention, the active medium, through which a forced circulation of the air to be treated is organized, consists of a stationary active mass that is produced in the form of a three-dimensional mesh network that is exposed to germicidal radiation that destroys organic pollutants that are possibly present in the air and trapped in the voids of said mass by impact against the inside walls that constitute its specific surface area (which constitute the photo-catalytic-action wall elements to which reference was made above).

For a dry-phase operation, the invention therefore proposes in particular an air purifier device characterized in that it comprises an active mass in the form of a three-dimensional mesh network that is exposed to a longitudinal source of germicidal radiation, as well as a ventilation system that forces the air to be treated to circulate through said mass primarily in the longitudinal direction of the longitudinal germicidal radiation source.

For an optimum use of the radiation source and a homogeneous distribution of the effects of the active mass, it is particularly advantageous to adopt a cylindrical configuration where the active mass that is subject to the circulation of the air to be treated forms a shell that surrounds the longitudinal source. Best advantage is then taken of germicidal radiation for a light source that emits in the ultraviolet range, but it can also provide, in combination or as a variant, in particular in combination, a germicidal action with a photo-catalytic action that helps ensure the destructive action of the pollutants within the active mass. The active mass is preferably itself produced, at least in part if not completely, such that on its specific surface, it consists of a material with photo-catalytic action in the presence of the radiation to which it is exposed on the path of the air that passes through it. In this way, the germicidal and photo-catalytic actions are combined in terms of an effective destruction of the organic pollutants that are contained in the inside air, whereby the first act by opening double bonds of the DNA molecules and the second act by helping to ensure the oxidation of the organic material.

The active mass can be based on different materials that are themselves known for other applications. In particular, metal fabrics are considered here, and more particularly those that are made of a framework of woven metal wire. All of the thus constituted mesh networks based on metal wires or fibers have the advantage of being easy to treat to obtain superficial photo-catalyst properties. A start is made in particular by deposition of a coating of photo-catalyst compound by quenching in a solution of this compound of fabrics, fibers or wires, which are then shaped or assembled in a three-dimensional multi-layer mass. Furthermore, knit fabrics have the capability, better than woven or non-woven fabrics, to adapt themselves to deformations that make it possible, for example, to tighten the mesh network elastically on a cooperating radiation-source central tube, or to fold the product on itself to obtain a multi-layer unit, or else to compress it more or less on itself to vary the density of the material that is subject to the impact or to photo-catalysis or to vary the mesh dimensions from one point to the next of the overall active mass.

Other materials to recommend in variant embodiments of the invention are represented by the porous materials in the mass. It is known, for example, that properties that are similar to those that are sought according to the invention in the metal fabrics or frameworks can be obtained from other types of porous masses.

From the standpoint of the role of the active mass in the process according to the invention, it will be noted that it is known that particles that are thrown at great speed on a target have a tendency to be retained on the surface of the latter. This is what is called the impact phenomenon. It is used in a known manner to collect living microorganisms, whereas according to the invention, it is used, on the contrary, so as to contribute to their destruction.

However, this is not the only action of which the invention takes advantage because of the impact of particles conveyed by the air on the inside walls of the active mass. The energy loss that tends to make these particles adhere to the active surface is already favorable to the effectiveness of the process, in particular in the case of a photo-catalytically-treated surface, but there is the added aspect that, on the one hand, the slowed particles remain trapped for a time in the voids in the active mass, although overall they are checked more than the air in their travel that is exposed to germicidal radiation, and there is the other added aspect that, on the other hand, the particles are weakened by these impacts against the inside walls of the active mass. In particular, these impacts tend to cause a break-up of relatively large particles and to shatter the particularly resistant shells of the microorganisms that are in particular spores, by thus allowing a germicidal radiation to reach them more easily at the core to alter growth and cellular division and to stop their reproduction.

As was already indicated, the photo-catalyst agent integrated into the active mass preferably consists of titanium dioxide. In combination with such a photo-catalyst, a germicidal radiation source that emits at approximately 250 to 270 nanometers is advantageously provided. The UVC radiation is very energetic, and it is capable of penetrating the voids of the active mass by radially passing through it in its entire thickness around an emitter tube that constitutes the longitudinal source, in particular when in the same period, the latter extends over a sufficient length so that the air passage time in the longitudinal direction parallel to the source ensures complete destruction of the polluting organic particles. On the other hand, it is easy to prevent the ultraviolet radiation from producing ozone, whereas the conditions of wavelengths that allow the activation of a titanium-dioxide-based catalyst are met. Also on the other hand, the desired germicidal activity is optimal in this wavelength range for viruses and common bacteria, and it is also optimal with regard to spores to the extent that resistance of their walls was reduced in advance by the mechanical action of the impacts undergone within the active mass.

So as to achieve an additional objective of the invention, which is to ensure that the exposure of the active mass to the radiation that illuminates it is complete and homogeneous in order to ensure the complete destruction of the particles that are temporarily trapped within, the invention advantageously provides that the mesh network, or mesh, occupies the entire section of a cylindrical shell that delimits the operational space of the device around the light source. This source is arranged in the device in the axis of an annular space that is occupied by the mesh, which makes it possible to illuminate the latter completely in a homogeneous manner.

From this standpoint, a particularly advantageous arrangement of the invention consists in completing the device by reflecting means of the radiation that tend to send it toward the active mass. It thus is possible, at the same time, to improve, on the one hand, the operational yield of the radiation emitted for the purposes of the process, in particular regarding the germicidal action, and to improve, on the other hand, its effectiveness in each cell or mesh of the active mass by helping it to be sent in all directions to strike the different walls with photo-catalytic properties.

The focus will now be more particularly on situations where it is preferred to operate in the wet phase rather than in the dry phase as above. While retaining a design of the equipment and operating conditions that essentially ensure the same combined actions of germicidal irradiation, photo-catalysis and impact, it is a matter of replacing the trapping of organic pollutants during destruction that is carried out in voids of the stationary active mass by a similar trapping that is carried out this time within a liquid mass that is mixed by the air to be treated and that is exposed to germicidal radiation, whereas it contains the solid wall elements for the impact and the photo-catalyst (advantageously on the same walls) in the form of particles that are kept dispersed in the liquid mass.

Advantageously in this case, the invention is translated into the form of an air purification process that consists in causing the air to be treated to circulate upon contact with a liquid mass in which particles, onto which the organic pollutants that are contained in this air are thrown, are dispersed, whereby said liquid mass is furthermore exposed to irradiation by a germicidal-action radiation that is activated by a photo-catalyst that is dispersed in the liquid mass. The photo-catalyst is preferably carried by the particles that are dispersed in the liquid mass and on which the pollutant impact phenomenon occurs.

So as to increase the contact surface area between pollutants and dispersed particles, to increase the irradiation surface area by the germicidal radiation source, and to improve the mixing with both pollutants and particles dispersed in the liquid mass, the circulation of the air is preferably carried out so as to drive the liquid mass in rotation on itself to create a vortex within the latter. An improved effectiveness of the photo-catalytic treatment results, as much by the increase in the passage time of the air upon contact with the liquid mass, resulting from the vortex, as by the increase in the dwell time of the pollutants under irradiation, resulting from the trapping of said pollutants within the liquid mass.

The invention also has as its object an air purification device that comprises, on one hand, a container that holds a liquid mass into which particles are dispersed, and, on the other hand, a source of illumination of this liquid mass by a germicidal radiation, preferably an ultraviolet radiation of the “C” range, and, furthermore, a ventilation system that forces the air to be treated to circulate upon contact with the liquid mass to create a vortex in the latter. The pollutants that are contained in the air that is introduced into such a purification device are entrained by this vortex to be thrown on the particles that are dispersed in the liquid mass, and they are trapped in the latter where they undergo a photo-catalytic treatment that is triggered by the germicidal radiation source and activated by a photo-catalyst that is dispersed in this liquid mass.

So as to promote the creation and preservation of this vortex, the invention calls for the container that holds the liquid mass to preferably be conical in shape, with a portion of larger diameter located on the side through which the air to be treated is introduced into the container. In addition, the invention calls for the air to be treated to be preferably introduced into this container tangentially to the walls of the latter so as to trigger the vortex action as soon as the air is introduced into the purification device according to the invention.

In this case, for an optimum irradiation of the liquid in its entire volume, the germicidal radiation source is preferably placed along the axis of revolution of the cone that is formed by the container that holds the liquid mass.

On the other hand, it is important to help ensure a long dwell time of the air to be treated upon contact with the particles that are dispersed into the liquid mass, both for the effectiveness of the impact phenomenon and for that of the photo-catalytic action. For this purpose, the invention, in particular, calls for the air to be treated to be injected into this container by a tube that empties into the portion of larger diameter of the latter and for it to be evacuated from said container also in the vicinity of the portion of larger diameter of the latter. Thus, under the action of the vortex that is triggered by its tangential injection into the container that holds the liquid mass, the air to be treated is forced, by the geometry of the collecting container, to circulate in a first step toward the portion of smaller diameter of the container (i.e., toward the tip of the cone that forms this container), and then in a second step, to circulate again toward the portion of large diameter of this container. The dwell time in contact with the particles that are dispersed into the liquid mass is thus increased, which helps to ensure a greater effectiveness of the treatment.

The ventilation system is, for its part, sized so that the speed that it communicates to the air to be treated is sufficient so that the latter, by its tangential injection into the container that holds the liquid mass, can generate the desired vortex.

The particles that are dispersed within the liquid mass preferably consist of titanium dioxide, at least on the surface. It can be a matter in particular of particles of polyethylene (inert material) that are coated by titanium dioxide, even though it will often be preferable to select particles that incorporate titanium dioxide in the mass, so as to preserve the catalyst action independently of the wear of the particles over time.

In the practical implementation of the invention, the dry purifier as defined above in general will have the advantage of effectively removing almost all of the organic pollutants from strongly polluted ambient air, whereas the wet-phase process will be reserved for finishing treatments.

The result is that a particularly elaborate air purification process according to the invention will comprise two cascade purifiers, one of each type, preferably with the purifier on a stationary three-dimensional network arranged upstream from the purifier with dispersed particles in a liquid environment on a circuit of air, whereby the air to be treated passes through them in series.

The invention thus makes it possible, by the combination of the two primary treatment paths that have been described, to ensure an optimum effectiveness of the elimination of the microorganisms in the polluted air. It extends to an air purification device that combines the two trapping purifiers respectively in dry phase and in liquid phase. Of this complete device, a description will be given later of a particularly advantageous embodiment where the two purifiers are mounted side by side in a configuration that has the advantage of being compact and taking up little space for optimum conditions of air circulation, with full allowance for the specific features of each of the two treatment environments.

Other characteristics and advantages of the invention will emerge from reading the following description of different embodiments of the latter, which description is illustrated by:

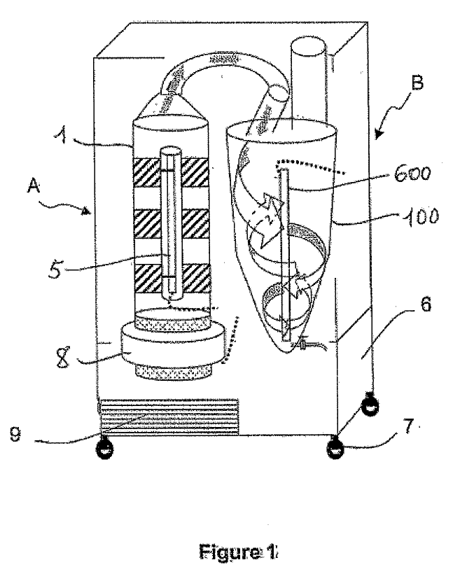

FIG. 1, which is a diagrammatic view of an air purification device according to the invention in its preferred embodiment according to which a liquid-phase trapping purifier is combined with a dry-phase trapping purifier,

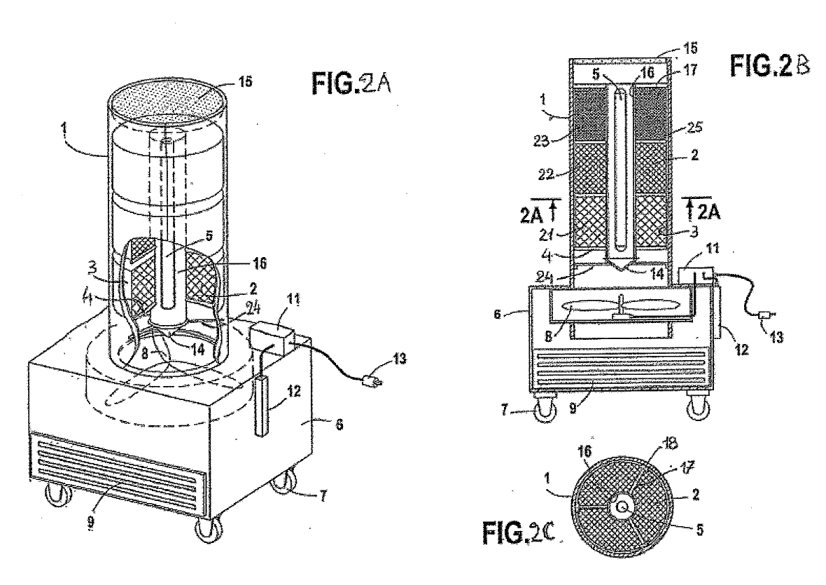

FIGS. 2A, 2B, 2C, which are respectively a diagrammatic perspective view and a diagrammatic view in longitudinal section of a dry-phase trapping air purification device,

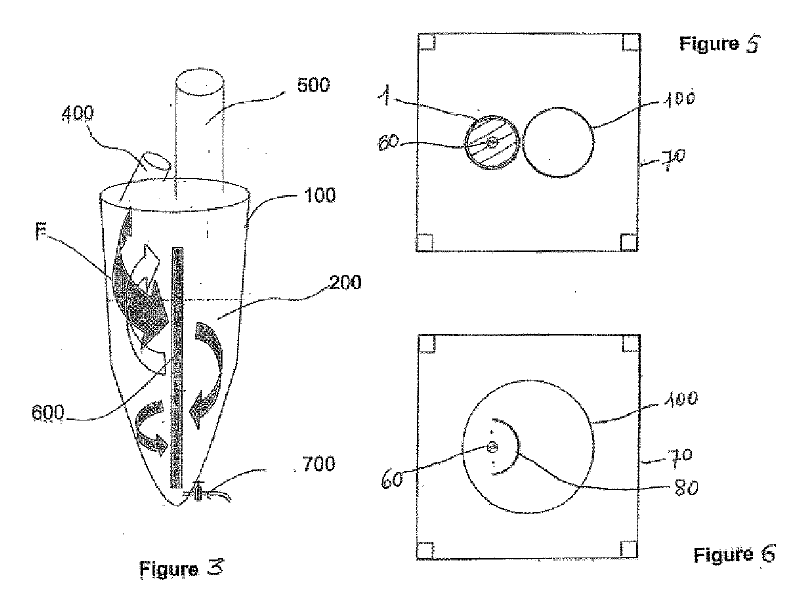

FIG. 3, which is a diagrammatic perspective view of a liquid-phase trapping air purification device,

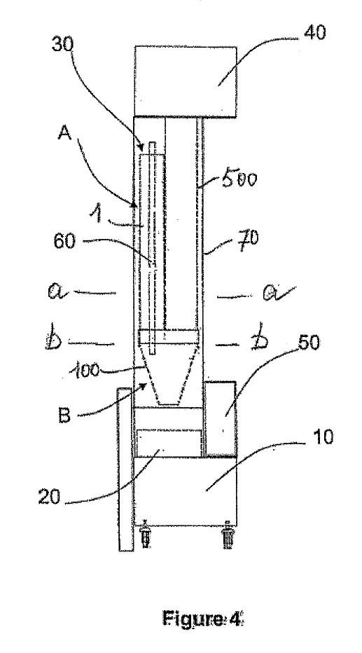

FIG. 4 is a diagrammatic cutaway view of an embodiment of an air purification device according to the invention in which a common source of germicidal radiation is used for the two air purifiers that it comprises,

and FIGS. 5 and 6 are diagrammatic representations of the device of FIG. 4 in cross-section along a-a or b-b of FIG. 4 respectively.

For more clarity, the same elements are referred to by the same references below.

With reference to FIG. 1, an air purification device according to the invention in its preferred embodiment comprises a dry-phase trapping air purifier A and a liquid-phase trapping air purifier B.

It is seen that the purification device according to the invention is implemented as a mobile unit and placed on a lower box 6 that is mounted on small wheels 7, which makes it possible to move it easily from one room to another or from one location to another in the same room. The lower box 6 contains the ventilation system at the intake into the purifier A, whereas the purified air is evacuated from the purifier B.

The description now made with reference to FIGS. 2A, 2B, and 2C relates to a dry-phase trapping air purifier that can certainly be adapted to constitute the purifier A of FIG. 1, but that is built to be used in an independent purifier.

Its construction in its entirety preserves a geometry with circular revolution symmetry around a central longitudinal axis that is occupied by a radiation source 5. The purifier is delimited by a tubular shell 1, here cylindrical in shape with a circular section, which is arranged, vertically relative to the ground, stationary on the box 6. This shell, rigid and mechanically resistant, typically consists of plastic material or stainless steel. It is surrounded by a sheath 3, made of, for example, sheet aluminum, whose purpose is to reflect the ultraviolet rays in the wavelength range of the source that is used.

The shell 1 encloses a mesh 2 that constitutes the destructive active mass of the organic pollutants. It is a matter of a three-dimensional mesh network, based on metal wire, which fills the internal volume in the shell 1. More specifically, it has been provided preferably to use a metal woven-wire fabric, which is folded into several layers on itself, for example by winding on itself in the manner of a metal household sponge In addition, the metal wire that is used was previously coated by a compound based on titanium dioxide. In this way, the active mass has a specific surface area with photo-catalytic action that causes an oxidative decomposition of the pollutant molecules that are usually present in air. Under the operating conditions of the device, the mesh dimensions are selected loosely enough to allow a free diffusion of ultraviolet radiation within the entire active mass.

In the center of the purifier, in the axis of the shell 1, the germicidal radiation source 5 represents the other essential element of the dry-phase trapping air purifier. It consists, more particularly, of a longitudinal lamp that emits ultraviolet radiation that is surrounded by a protective tube 16 that is made of quartz to be transparent to the UV rays. The lamp 5 is held in the vertical axis of the tube 16 by any means known in the art, whereby the tube 16 is itself held by star-shaped support crosspieces whose branches 17 are welded on the shell 1. The assembly is selected to be emitting within the UVC wavelength range, as it is known to obtain it by, for example, a mercury lamp.

The active mass mesh 2 extends entirely around the tube 16 that envelops the UV lamp and radially from the latter up to the reflective sheath 3 that surrounds the shell 1 on the inside. From place to place, its material strands are welded onto vertical rods 18, optionally also on the sheath 3. The mesh is thus prevented from sagging, and also air gaps that pass through the purifier from bottom to top are prevented from being formed. In the longitudinal direction, the active mass is distributed in three beds that all three cover the level of treatment from the input of the air flow through the lower end of the shell up to its output at the upper end, whereby the distance between input and output is enough to ensure the desired degree of purification and to prevent, as soon as it is released into the atmosphere, the clean air from mixing with dirty air that has not yet been treated.

Diagrammatically, it was brought out in FIGS. 2B and 2C that the constituent mesh of the active mass is not uniform. It exhibits a mesh dimension variation gradient that decreases along the path of the air. In practical implementation, it is observed that in the distribution into three beds, the mesh is very loose in the lower bed 21, then tight in the intermediate bed 22, and very tight in the upper bed 23. Such a gradient in the longitudinal direction of the air circulation is advantageous in that large particles that are present in the air at the input of the lower bed can pass through the mesh of the latter and reach the upper bed only after having fractionated into small particles, which can be treated there without causing its clogging.

In certain embodiments of the invention, it is also possible to provide a transverse gradient by compressing radially the mesh toward the UV lamp in a compromise between the passage speed of the air, the filling density of walls that are accessible to impact, and the void density that is accessible to the penetration of UV rays.

In the successive stages of the active mass 2, an empty space is provided between the two successive mesh beds on a layer height that is considerably larger than was illustrated at 25 for the example of FIG. 2B. The space constituted by the empty mesh layer offers a turbulent flow zone to the air in circulation. While preserving a direction of circulation overall in the longitudinal direction of the treatment unit, a mixing of air circulation jets is thus induced at each empty mesh layer so as to help ensure that the air that was treated thoroughly along the lamp then, once treated, moves on, close to the outside shell, and vice versa. In addition, the same layer of air between the mesh beds is allowed to pass directly through the UV rays, those that emanate from the lamp itself, but also those that return after reflection on the sheath 3. Also because of the reflections inside the mesh, overall, a multi-directional diffusion of the UV rays is created toward the adjacent zones of the two adjacent mesh beds.

In the lower portion of the shell 1, in the conveyor of the ventilator 8, a deflector screen 14 is inserted on the path of the air so as to prevent it from penetrating in the immediate vicinity of the UV lamp, without first passing through a zone filled with active mass mesh. This deflector is conical in shape, with a point directed downward. It covers the entire width of the central tube 16. It thus is used to deflect the air flow outside of the zone occupied by the central light source by directing it toward the intake of the active mass. From the standpoint of mechanical assembly, the deflector 14 is carried in a stationary way by a support 24 with three rods in the shape of a star, which is welded directly onto the outside shell that is separate from the star-shaped crosspiece 4 that supports the mesh of the lower active mass bed 21.

At the upper end of the shell 1, a filter 15 is provided that makes it possible to hold the mineral particles that thus usefully supplement the action of the active mass under irradiation, to the extent where only the particles of organic nature have been destroyed during the passage of the active mass. It will be noted that this type of filter is advantageous only in the case where the purifier is designed for an independent operation. Actually, if the dry-phase treatment is followed by a wet-phase treatment, the mineral particles will be held in the liquid medium that is used in the downstream purifier, which thus will play the role of final filter.

As the air moves into the mesh beds that constitute the active mass, the particles that it entrains undergo a purification process that results from the synergetic combination of three phenomena that combine their potentializing actions so as to ensure the destruction of pollutants that are organic in nature. The germicidal action of the radiation is reflected essentially on the microorganism particles, while these particles like the polluting particles undergo the action of the impact process on the inside walls in the mesh and thus see their structural and functional characteristics altered, and at the same time, the photo-catalytic action attacks by oxidation all of the molecules that are sensitive thereto, especially when they are temporarily trapped in the empty cells of the mesh.

Now comes the description of FIG. 3, illustrating the composition of a wet purifier according to the invention, by taking into consideration that it is designed to be coupled to a dry purifier preceding it on the path of the air to be treated.

Such a purifier comprises a container 100 that holds a liquid mass 200 (actually water) in which solid particles in suspension are dispersed. The container 100 has a conical shape whose tip is directed downward. The air is allowed into the purifier via a tube 400 that empties into the chamber that contains the liquid mass, above the water level. It is extracted at the same level via a tube 500 that opens into the surrounding atmosphere. A purge spigot 700 is provided at the bottom of the container. It is used to evacuate the water to replace it, partially or completely, when it becomes encumbered with mineral particles that are not destroyed by the process or when the catalyst particles are consumed and too spent.

The air is injected into the container 100 via the intake tube 400 so as to follow a lamellar path that is coiled in a helix by licking the walls of the container, as it is illustrated by the arrows F in FIG. 3. The liquid mass is put into motion by this air flow. A vortex that ensures a condenser and uniform mixing of the liquid mass is thus generated, entraining with it the particles that it contains. To help ensure the production of the vortex, the intake tube 400 empties essentially tangentially to the inside wall of the container 100 at its upper portion of larger diameter.

The advantage of the mixing of the particles under the action of this vortex is multiple. It is ensured that the air describes a long path through the liquid mass, first following a helix by descending, then following another helix by rising. As for the particles, they are continuously brought down from one location to another of the treatment tank, by presenting their entire surfaces in turn to the impact of pollutants during destruction, by periodically coming close to the radiation source for what is their photo-catalytic action. The dwell time of the air in the active medium as well as the multiplicity of particles that it encounters there help to ensure the impact phenomenon, and there is the added aspect that the vortex imparts additional energy to the pollutants during the destruction that it causes, which also helps to ensure this impact phenomenon.

The organic pollutants that are reduced and/or weakened because of the impact phenomenon are subjected to a photo-catalyzed germicidal treatment for the entire time that they are trapped within the liquid mass. For this purpose, the purifier comprises a germicidal radiation source, constituted by a tube 600 that emits in the “C” range of the ultra-violet spectrum that is arranged vertically in the tank 100. If necessary, the walls of the tank are made of a transparent material, such that the user of the purifier can observe the operation of the vortex and see the state of the liquid medium.

The particles that are dispersed in the liquid active mass are advantageously produced in a chemically inert polymer material, preferably based on polyethylene, whose surface is coated by a compound based on titanium dioxide. Light because of their low density, they can easily remain dispersed within the liquid mass. Inert in their mass, they have only a very slight risk of reaction with the pollutants that are trapped within the liquid mass in the event that their photo-catalytic coating becomes damaged over time. However, it will often be preferable, as a variant, to constitute the particles of a material that contains titanium dioxide in the mass. This is favorable in particular in consideration of the possibilities of superficial wear of the particles.

In a purification device according to the invention that combines a dry-phase trapping air purifier A and a liquid-phase trapping air purifier B as it is shown in FIG. 1, the box 6 is advantageously equipped with small pivoting wheels 7, which makes it possible to bring the unit easily to all desired points of the room whose inside air is to be purified. The box houses a ventilator 8 that draws in the air from the inside of the room, through the louvers of the air intake 9 that are provided on a lateral surface of the box. At the top of the dry-phase purifier, the air preserves an adequate speed to be allowed into the wet-phase purifier and to create the vortex action there.

Another configuration of a device according to the invention that combines the two types of purifiers is applied in the embodiment that is illustrated by FIGS. 4, 5 and 6. In this case, the two purifiers are no longer truly arranged side by side, but rather one below the other, and they operate with a single source of radiation, which is common to them.

In the particular case that is described, the cone of the wet-phase purifier is arranged lower than the dry-phase purifier. One and the other remain centered around a vertical axis, but they are not on the same axis. This allows a compact arrangement, where above the tank 100, the output tube of the purified air 500 passes beside the dry-phase purifier, alongside the latter.

The UV radiation source consists of a tube 60 of which it is seen in FIG. 4 that it passes all along the shell 1 that contains the three-dimensional active mass in the purifier A and that beyond, in the low portion of the device, it penetrates into the tank 100 that contains the liquid mass with dispersed particles of the purifier B. Taking into account this arrangement, the circulation of the air in the three-dimensional mass is no longer from bottom to top but from top to bottom.

The air to be treated is drawn into a box 10 that is located in the low portion of the device. It is fed back through a ventilator 20 into a chamber 70 that constitutes a closed shell, here with a square section, all around the two purifiers and the tube 500. Its speed of ascent is adequate so that without disturbing the intake in the vortex, it is led up to the top of the column of the purifier A, at 30 in FIG. 4. From the purifier A, it passes directly into the liquid-phase trapping purifier B, located below. It goes out via the tube 500, which passes through the chamber 70 in an airtight manner to rediffuse it into the atmosphere from a collector 40, optionally equipped with an extractor.

FIG. 4 shows the presence of a water reserve at 50. Water is taken from it automatically in order to add it to compensate for the operational losses and to keep the liquid mass at a constant, since it is desirable for the stability of the vortex. Finally, it is observed from FIG. 6 that the UV tube is in a position that is off-center relative to the tank 100 where it penetrates to constitute there the radiation source of the purifier B and that it is protected from too direct an intake of the air flow via a deflector 80.

1. A method for air purification that consists essentially in forcing the air to be treated to circulate through an active medium exposed to a germicidal radiation in the presence of a photo-catalytic material contained in said medium which is active for destroying the microorganisms possibly present in said air that are retained in the active medium as reduced in size and/or weakened by impact on wall elements that are present for this purpose in the active medium.

2. A method according to claim 1, in which said active medium consists of an active mass that is produced in the form of a three-dimensional network of walls with a photo-catalytic surface that is exposed to said germicidal radiation, whereby the organic pollutants that are possibly present in the air in circulation through said active mass are trapped in the voids of the latter by impact on the inside walls that constitute its specific surface.

3. A method according to claim 2, in which said active mass consists of a multi-layer metal framework with a photo-catalytic coating, in particular based on titanium dioxide, which is arranged around a longitudinal germicidal radiation source, in particular constituted by an ultraviolet radiation emitting tube, preferably in the UVC range, and in which the air circulation is ensured in a longitudinal direction parallel to the axis of the longitudinal source.

4. A method according to claim 1, in which said active medium consists of a liquid mass in which particles with photo-catalytic activity are dispersed and which is exposed to a germicidal radiation which, in the presence of said particles with photo-catalytic activity, has a destructive action for the organic pollutants that are possibly present in the air in circulation through said liquid mass and are trapped in said mass by impact on particles with a solid wall contained therein as a suspension.

5. A method according to claim 1, in which the air to be treated is allowed into the liquid mass so as to generate a vortex there.

6. An air purification device that comprises at least one purifier that comprises means for circulation of the air to be treated through an active mass that contains a photo-catalytic material and solid wall elements and exposed to a germicidal UV radiation source with a destructive action on the organic pollutants that are possibly present in the air in circulation and have been reduced in size and/or weakened by impact on said solid wall elements and trapped in their vicinity in said active mass.

7. A device according to claim 6 that comprises at least one dry-phase purifier that comprises means for forcing the air to be treated to circulate through said active mass and in which said active mass is produced in the form of a three-dimensional network of walls with a photo-catalytic surface that is arranged around said germicidal radiation source and whereby the air circulation is ensured parallel to the axis of said source.

8. A device according to claim 7, in which said active mass is surrounded by a sheath that is reflective for the radiation emitted by said source.

9. An air purifier device according to claim 7, in which said mass is distributed longitudinally into several mesh beds with photo-catalytic walls that are separated by empty mesh layers that provide the circulating air with turbulent flow zones and/or in which said active mass has a gradient of mesh dimensions that ranges from a looser mesh to a tighter mesh in the direction of circulation of the air.

10. An air purification device according to claim 6, comprising at least one wet-phase purifier in which said active mass is produced in liquid form in a container that holds particles with photo-catalytic activity dispersed into said mass that also constitute said wall elements for the impact of organic pollutants and in which air intake means that cause the formation of a vortex in said liquid mass are provided.

11. An air purification device according to claim 7 that comprises means for forcing the air to be treated to circulate first through said dry-phase purifier and then through a wet-phase purifier, whereby the wet-phase purifier is preferably arranged vertically below the dry-phase purifier and the latter contains a vertical radiation source that penetrates into the wet-phase evaporator to constitute its radiation source there.
